SEQUENCE LISTING (4 sequences)

SEQ ID NO: 1 - protein, 267 aa, Homo sapiens
MGSACIKVTKYFLFLFNLIFFILGAVILGFGVWILADKSSFISVLQTSSSSLRMGAYVFIGVGAVTMLMGFLGCIGAVNEVRCLLGLYFAFLLLILIAQVTAGALFYFNMGKLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENLGIILGVGVGVAIIELLGMVLSICLCRHVHSEDYSKVPKY

SEQ ID NO: 2 - protein, 242 aa, Homo sapiens
MGSACIKVTKYFLFLFNLIFFILGAVILGFGVWILADKSSFISVLQTSSSSLRMGAYVFIGVGAVTMLMGFLGCIGAVNEVRCLLGLLKQEMGGIVTELIRDYNSSREDSLQDAWDYVQAQVKCCGWVSFYNWTDNAELMNRPEVTYPCSCEVKGEEDNSLSVRKGFCEAPGNRTQSGNHPEDWPVYQEGCMEKVQAWLQENLGIILGVGVGVAIIELLGMVLSICLCRHVHSEDYSKVPKY

SEQ ID NO: 3 - DNA, 1640 bp, Homo sapiens
ggcctgccgagtccgcggcgttccccggctgcagccgggagggggccgaggagtgactgagccccgggctgtgcagtccgacgccgactgaggcacgagcgggtgacgctgggcctgcagcgcggagcagaaagcagaacccgcagagtcctccctgctgctgtgtggacgacacgtgggcacaggcagaagtgggccctgtgaccagctgcactggtttcgtggaaggaagctccaggactggcgggatgggctcagcctgtatcaaagtcaccaaatactttctcttcctcttcaacttgatcttctttatcctgggcgcagtgatcctgggcttcggggtgtggatcctggccgacaagagcagtttcatctctgtcctgcaaacctcctccagctcgcttaggatgggggcctatgtcttcatcggcgtgggggcagtcactatgctcatgggcttcctgggctgcatcggcgccgtcaacgaggtccgctgcctgctggggctgctgaagcaggagatgggcggcatcgtgactgagctcattcgagactacaacagcagtcgcgaggacagcctgcaggatgcctgggactacgtgcaggctcaggtgaagtgctgcggctgggtcagcttctacaactggacagacaacgctgagctcatgaatcgccctgaggtcacctacccctgttcctgcgaagtcaagggggaagaggacaacagcctttctgtgaggaagggcttctgcgaggcccccggcaacaggacccagagtggcaaccaccctgaggactggcctgtgtaccaggagggctgcatggagaaggtgcaggcgtggctgcaggagaacctgggcatcatcctcggcgtgggcgtgggtgtggccatcatcgagctcctggggatggtcctgtccatctgcttgtgccggcacgtccattccgaagactacagcaaggtccccaagtactgaggcagctgctatccccatctccctgcctggcccccaacctcagggctcccaggggtctccctggctccctcctccaggcctgcctcccacttcactgcgaagaccctcttgcccatcctgactgaaagtagggggctttctggggcctagcgatctctcctggcctatccgctgccagccttgagccctggctgttctgtggttcctctgctcaccgcccatcagggttctcttagcaactcagagaaaaatgctccccacagcgtccctggcgcaggtgggctggacttctacctgccctcaagggtgtgtatattgtataggggcaactgtatgaaaaattggggaggagggggccgggcgcggtggctcacgcctgtaatcccagcactttgggaggccgaggcgggtggatcacgaggtcaggagatcgagaccatcctggctaacatggtgaaaccccgtctctactaaaaatacaaaaaaaatttagccgggcgcggtggcgggcacctgtagtcccagctacttgggaggctgaggcaggagaatggtgtgaacccgggagcggaggttgcagtgagctgagatcgtgctactgcactccagcctgggggacagaaagagactccgtctcaaaaaaaaaaaaaaaaaaaaaaaaaa

SEQ ID NO: 4 - DNA, 2212 bp, Homo sapiens
gcagtccgacgccgactgaggcacgagcgggtgacgctgggcctgcagcgcggagcagaaagcagaacccgcagagtcctccctgctgctgtgtggacgacacgtgggcacaggcagaagtgggccctgtgaccagctgcactggtttcgtggaaggaagctccaggactggcgggatgggctcagcctgtatcaaagtcaccaaatactttctcttcctcttcaacttgatcttctttatcctgggcgcagtgatcctgggcttcggggtgtggatcctggccgacaagagcagtttcatctctgtcctgcaaacctcctccagctcgcttaggatgggggcctatgtcttcatcggcgtgggggcagtcactatgctcatgggcttcctgggctgcatcggcgccgtcaacgaggtccgctgcctgctggggctgtactttgctttcctgctcctgatcctcattgcccaggtgacggccggggccctcttctacttcaacatgggcaagctgaagcaggagatgggcggcatcgtgactgagctcattcgagactacaacagcagtcgcgaggacagcctgcaggatgcctgggactacgtgcaggctcaggtgaagtgctgcggctgggtcagcttctacaactggacagacaacgctgagctcatgaatcgccctgaggtcacctacccctgttcctgcgaagtcaagggggaagaggacaacagcctttctgtgaggaagggcttctgcgaggcccccggcaacaggacccagagtggcaaccaccctgaggactggcctgtgtaccaggagggctgcatggagaaggtgcaggcgtggctgcaggagaacctgggcatcatcctcggcgtgggcgtgggtgtggccatcatcgagctcctggggatggtcctgtccatctgcttgtgccggcacgtccattccgaagactacagcaaggtccccaagtactgaggcagctgctatccccatctccctgcctggcccccaacctcagggctcccaggggtctccctggctccctcctccaggcctgcctcccacttcactgcgaagaccctcttgcccatcctgactgaaagtagggggctttctggggcctagcgatctctcctggcctatccgctgccagccttgagccctggctgttctgtggttcctctgctcaccgcccatcagggttctcttagcaactcagagaaaaatgctccccacagcgtccctggcgcaggtgggctggacttctacctgccctcaagggtgtgtatattgtataggggcaactgtatgaaaaattggggaggagggggccgggcgcggtggctcacgcctgtaatcccagcactttgggaggccgaggcgggtggatcacgaggtcaggagatcgagaccatcctggctaacatggtgaaaccccgtctctactaaaaatacaaaaaaaatttagccgggcgcggtggcgggcacctgtagtcccagctacttgggaggctgaggcaggagaatggtgtgaacccgggagcggaggttgcagtgagctgagatcgtgctactgcactccagcctgggggacagaaagagactccgtctcaaaaaaaaaaaaaaaaaaaaaaaaaattggggagggaagggcgttagataaggcactctgggctgtcaggagactgcctactgggtgggtcaacttatttcccactatgacatttatatctttatttttcacaattatattgaaggcagtgggagaggggagagggtgggttttacctgatattaaggggtggcaccccttccccaggcctgatgcaggtccatctagacagctcagattagcctaggccatgccctagggacacggcctaggggagctgggctggagggggtctgcctgggtaaggggatctggcttggccctgagctgcttagggacaggtgagtccttgggggcatggcacagagctggtcctgtattctccagggtccggagctggccaggggcggggaggaggaggatggcttctcctgggagaaggtgggctggcctttcttggcaagcggtcctctggcccctggggagtggggtgggtgaggctgtgccttgtagggggagaggggagggggctttgtgtgcaccattctgtaaaacacactcaagattctaagactattaaagaggatttataaca

(19) United States
(12) Patent Application Publication
(10) Pub. No.: US 2022/0119489 A1
(43) Pub. Date: Apr. 21, 2022

(54) COMPOSITIONS AND METHODS FOR TREATING MUSCULAR DYSTROPHY AND RELATED DISORDERS

(71) Applicant: CHILDREN'S MEDICAL CENTER CORPORATION, Boston, MA (US)

(72) Inventor: Emanuela GUSSONI, Boston, MA (US)

(73) Assignee: CHILDREN'S MEDICAL CENTER CORPORATION, Boston, MA (US)

(21) Appl. No.: 17/425,272

(22) PCT Filed: Nov. 25, 2019
(86) PCT No.: PCT/US19/63023

§ 371 Date: Jul. 22, 2021

Related U.S. Application Data
(60) Provisional application No. 62/797,007, filed on Jan. 25, 2019.

(51) Int. Cl. C07K 14/705; C12N 15/86; A61K 31/573; A61K 31/19; A61K 31/585; G01N 33/68; A61P 21/00

(52) U.S. Cl. CPC C07K 14/70596; C12N 15/86; A61K 31/573; A61K 31/19; C12N 2750/14143; G01N 33/6893; A61P 21/00; G01N 2333/70596; G01N 2800/10; A61K 31/585

(57) ABSTRACT

The present invention features compositions and methods featuring CD82 for treating muscular dystrophies and related disorders. In one aspect, the invention provides a method of preserving or increasing muscle function in a dystrophic cell, the method involving contacting the cell with a CD82 polypeptide or a polynucleotide encoding a CD82 polypeptide.

### CROSS-REFERENCE TO RELATED APPLICATION

This application claims the benefit of the following U.S. Provisional Application No. 62/797,007, filed Jan. 25, 2019, the entire contents of which are incorporated herein by reference.

### STATEMENT OF RIGHTS TO INVENTIONS MADE UNDER FEDERALLY SPONSORED RESEARCH

This invention was made with government support under Grant No. 1R01AR069582 awarded by the National Institutes of Health. The government has certain rights in the invention.

### BACKGROUND OF THE INVENTION

Muscular dystrophies are muscle degenerative diseases in which the muscle at first forms normally, but starts to degenerate faster than it can be repaired. The most common form of muscular dystrophy is Duchenne Muscular Dystrophy (DMD) representing over 90% of the diagnosed cases. DMD is a progressive and fatal muscle degenerating disease caused by a dystrophin deficiency. The predominant muscle dystrophin isoform is translated from the largest gene in the human genome. The gene encodes a large protein of 427 KDa that positions just inside of the sarcolemmal membrane and links the internal cytoskeleton with the muscle cell membrane. This linkage is vital to maintaining muscle membrane integrity during repeated cycles of cell contraction. Almost all known human dystrophin mutations that cause DMD typically result in the loss or degradation of the dystrophin protein at the sarcolemmal membrane.

Currently, there are no effective long-term therapies for treating DMD, the most common form of the disease, which affects approximately 1 in every 3,500 males born in the United States. Effective suppression of the primary pathology observed in DMD is critical for treatment.

### SUMMARY OF THE INVENTION

As described below, the present invention features compositions and methods for treating muscular dystrophies and related disorders.

In one aspect, the invention provides a method of preserving or increasing muscle function in a dystrophic cell, the method involving contacting the cell with a CD82 polypeptide or a polynucleotide encoding a CD82 polypeptide.

In another aspect, the invention provides a method for increasing and/or enhancing myofiber structure in a muscle cell or muscle progenitor cell, the method involving increasing the expression of a CD82 polypeptide or polynucleotide in a muscle cell or a muscle progenitor cell, where increasing the expression of a CD82 polypeptide or polynucleotide increases and/or enhances the myofiber structure of the cell.

In another aspect, the invention provides a method of repairing a cell membrane, the method involving increasing the expression of a CD82 polypeptide or polynucleotide in a muscle cell or a muscle progenitor cell, wherein increasing the expression of a CD82 polypeptide or polynucleotide repairs the muscle cell membrane.

In yet another aspect, the invention provides a method for increasing muscle cell membrane integrity, the method involving increasing the expression of CD82 in a muscle cell or a muscle progenitor cell, where increasing the expression of CD82 increases or enhances the muscle cell membrane integrity.

In still another aspect, the invention provides a method of treating a muscular dystrophy in a subject, the method involving administering to the subject an effective amount of a CD82 polypeptide or a polynucleotide encoding a CD82 polypeptide.

In yet another aspect, the invention provides a method for treating muscular dystrophy (MD), the method involving administering to a subject having or suspected of having MD an effective amount of a compound that increases CD82 polypeptide or polynucleotide expression. In one embodiment, the compound is sodium pyruvate, dexamethasone, or oxandrolone. In another embodiment, the method is performed in vitro or ex vivo.

In yet another aspect, the invention provides a mammalian expression vector having a promoter operably linked to a polynucleotide encoding human CD82. In some embodiments, the promoter is an actin promoter. In some embodiments, the vector is a lentiviral vector or adeno associated viral vector.

In yet another aspect, the invention provides a cell (e.g., mammalian cell) containing an expression vector having a promoter operably linked to a polynucleotide encoding human CD82. In some embodiments of this aspect, the cell is a muscle cell, a myofiber or a muscle progenitor cell.

In yet another aspect, the invention provides a pharmaceutical composition containing an effective amount of an expression vector having a promoter operably linked to a polynucleotide encoding a human CD82 polynucleotide or polypeptide.

In yet another aspect, the invention provides a method for detecting muscular dystrophy in a subject, the method involving detecting reduced levels of CD82 in a biological sample of a subject. In one embodiment, the biological sample is a tissue sample (e.g., muscle, skin, blood).

In yet another aspect, the invention provides a method for detecting CD82 polypeptide or polynucleotide levels in a sample derived from a subject having or suspected of having muscular dystrophy, the method involving contacting the sample with an antibody that specifically binds CD82 and detecting binding, thereby detecting CD82 levels in the sample. In one embodiment, a reduced level of CD82 in the sample relative to the CD82 in a sample derived from a subject not having or suspected of having muscular dystrophy is indicative of muscular dystrophy.

In various embodiments of the above aspects, or any other aspect of the invention delineated herein, the CD82 polynucleotide is present in a mammalian expression vector (e.g., an adeno associated viral vector or lentiviral vector). In some embodiments, the expression is driven by a muscle specific or inducible promoter. In some embodiments, the composition increases the expression of CD82 in one or more cells of the subject (e.g., muscle cells, satellite cells, myoblasts, myofibers, muscle side population cells, fibroblast cells, smooth muscle cells, stem cells, and mesenchymal stem cells).

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels.”

By “CD82 polypeptide” is meant a protein having at least about 85% amino acid sequence identity to NCBI Reference Sequence: NP_002222, NP_001020015, or a fragment thereof that reduces creatine kinase (CK) levels of a subject. Exemplary CD82 polypeptide sequences are reproduced below:

| | |
|---|---|
| 1 | mgsacikvtk yflflfnlif filgavilgf gvwiladkss fisvlqtsss slrmgayvfi |
| 61 | gvgavtmlmg flgcigavne vrcllglyfa fllliliaqv tagalfyfnm gklkqemggi |
| 121 | vtelirdyns sredslqdaw dyvqaqvkcc gwvsfynwtd naelmnrpev typcscevkg |
| 181 | eednslsvrk gfceapgnrt qsgnhpedwp vyqegcmekv qawlqenlgi ilgvgvgvai |
| 241 | iellgmvlsi clcrhvhsed yskvpky |

The sequence of NP_001020015 is reproduced below:

| | |
|---|---|
| 1 | mgsacikvtk yflflfnlif filgavilgf gvwiladkss fisvlqtsss slrmgayvfi |
| 61 | gvgavtmlmg flgcigavne vrcllgllkq emggivteli rdynssreds lqdawdyvqa |
| 121 | qvkccgwvsf ynwtdnaelm nrpevtypcs cevkgeedns lsvrkgfcea pgnrtqsgnh |
| 181 | pedwpvyqeg cmekvqawlq enlgiilgvg vgvaiiellg mvlsiclcrh vhsedyskvp |
| 241 | ky |

By “CD82 polynucleotide” is meant a nucleic acid molecule that encodes a CD82 polypeptide. Exemplary CD82 polynucleotide sequences are provided below. NM_001024844

| | |
|---|---|
| 1 | ggcctgccga gtccgcggcg ttccccggct gcagccggga gggggccgag gagtgactga |
| 61 | gccccgggct gtgcagtccg acgccgactg aggcacgagc gggtgacgct gggcctgcag |
| 121 | cgcggagcag aaagcagaac ccgcagagtc ctccctgctg ctgtgtggac gacacgtggg |
| 181 | cacaggcaga agtgggccct gtgaccagct gcactggttt cgtggaagga agctccagga |
| 241 | ctggcgggat gggctcagcc tgtatcaaag tcaccaaata ctttctcttc ctcttcaact |
| 301 | tgatcttctt tatcctgggc gcagtgatcc tgggcttcgg ggtgtggatc ctggccgaca |
| 361 | agagcagttt catctctgtc ctgcaaacct cctccagctc gcttaggatg ggggcctatg |
| 421 | tcttcatcgg cgtgggggca gtcactatgc tcatgggctt cctgggctgc atcggcgccg |
| 481 | tcaacgaggt ccgctgcctg ctggggctgc tgaagcagga gatgggcggc atcgtgactg |
| 541 | agctcattcg agactacaac agcagtcgcg aggacagcct gcaggatgcc tgggactacg |
| 601 | tgcaggctca ggtgaagtgc tgcggctggg tcagcttcta caactggaca gacaacgctg |
| 661 | agctcatgaa tcgccctgag gtcacctacc cctgttcctg cgaagtcaag ggggaagagg |
| 721 | acaacagcct ttctgtgagg aagggcttct gcgaggcccc cggcaacagg acccagagtg |
| 781 | gcaaccaccc tgaggactgg cctgtgtacc aggagggctg catggagaag gtgcaggcgt |
| 841 | ggctgcagga gaacctgggc atcatcctcg gcgtgggcgt gggtgtggcc atcatcgagc |
| 901 | tcctggggat ggtcctgtcc atctgcttgt gccggcacgt ccattccgaa gactacagca |
| 961 | aggtccccaa gtactgaggc agctgctatc cccatctccc tgcctggccc ccaacctcag |
| 1021 | ggctcccagg ggtctccctg gctccctcct ccaggcctgc ctcccacttc actgcgaaga |
| 1081 | ccctcttgcc catcctgact gaaagtaggg ggctttctgg ggcctagcga tctctcctgg |
| 1141 | cctatccgct gccagccttg agccctggct gttctgtggt tcctctgctc accgcccatc |
| 1201 | agggttctct tagcaactca gagaaaaatg ctccccacag cgtccctggc gcaggtgggc |
| 1261 | tggacttcta cctgccctca agggtgtgta tattgtatag gggcaactgt atgaaaaatt |
| 1321 | ggggaggagg gggccgggcg cggtggctca cgcctgtaat cccagcactt tgggaggccg |
| 1381 | aggcgggtgg atcacgaggt caggagatcg agaccatcct ggctaacatg gtgaaacccc |
| 1441 | gtctctacta aaaatacaaa aaaaatttag ccgggcgcgg tggcgggcac ctgtagtccc |
| 1501 | agctacttgg gaggctgagg caggagaatg gtgtgaaccc gggagcggag gttgcagtga |
| 1561 | gctgagatcg tgctactgca ctccagcctg ggggacagaa agagactccg tctcaaaaaa |
| 1621 | aaaaaaaaaa aaaaaaaaaa |
| 1 | gcagtccgac gccgactgag gcacgagcgg gtgacgctgg gcctgcagcg cggagcagaa |
| 61 | agcagaaccc gcagagtcct ccctgctgct gtgtggacga cacgtgggca caggcagaag |
| 121 | tgggccctgt gaccagctgc actggtttcg tggaaggaag ctccaggact ggcgggatgg |
| 181 | gctcagcctg tatcaaagtc accaaatact ttctcttcct cttcaacttg atcttcttta |
| 241 | tcctgggcgc agtgatcctg ggcttcgggg tgtggatcct ggccgacaag agcagtttca |
| 301 | tctctgtcct gcaaacctcc tccagctcgc ttaggatggg ggcctatgtc ttcatcggcg |
| 361 | tgggggcagt cactatgctc atgggcttcc tgggctgcat cggcgccgtc aacgaggtcc |
| 421 | gctgcctgct ggggctgtac tttgctttcc tgctcctgat cctcattgcc caggtgacgg |
| 481 | ccggggccct cttctacttc aacatgggca agctgaagca ggagatgggc ggcatcgtga |
| 541 | ctgagctcat tcgagactac aacagcagtc gcgaggacag cctgcaggat gcctgggact |
| 601 | acgtgcaggc tcaggtgaag tgctgcggct gggtcagctt ctacaactgg acagacaacg |
| 661 | ctgagctcat gaatcgccct gaggtcacct acccctgttc ctgcgaagtc aagggggaag |
| 721 | aggacaacag cctttctgtg aggaagggct tctgcgaggc ccccggcaac aggacccaga |
| 781 | gtggcaacca ccctgaggac tggcctgtgt accaggaggg ctgcatggag aaggtgcagg |
| 841 | cgtggctgca ggagaacctg ggcatcatcc tcggcgtggg cgtgggtgtg gccatcatcg |
| 901 | agctcctggg gatggtcctg tccatctgct tgtgccggca cgtccattcc gaagactaca |
| 961 | gcaaggtccc caagtactga ggcagctgct atccccatct ccctgcctgg cccccaacct |
| 1021 | cagggctccc aggggtctcc ctggctccct cctccaggcc tgcctcccac ttcactgcga |
| 1081 | agaccctctt gcccatcctg actgaaagta gggggctttc tggggcctag cgatctctcc |
| 1141 | tggcctatcc gctgccagcc ttgagccctg gctgttctgt ggttcctctg ctcaccgccc |
| 1201 | atcagggttc tcttagcaac tcagagaaaa atgctcccca cagcgtccct ggcgcaggtg |
| 1261 | ggctggactt ctacctgccc tcaagggtgt gtatattgta taggggcaac tgtatgaaaa |
| 1321 | attggggagg agggggccgg gcgcggtggc tcacgcctgt aatcccagca ctttgggagg |
| 1381 | ccgaggcggg tggatcacga ggtcaggaga tcgagaccat cctggctaac atggtgaaac |
| 1441 | cccgtctcta ctaaaaatac aaaaaaaatt tagccgggcg cggtggcggg cacctgtagt |
| 1501 | cccagctact tgggaggctg aggcaggaga atggtgtgaa cccgggagcg gaggttgcag |
| 1561 | tgagctgaga tcgtgctact gcactccagc ctgggggaca gaaagagact ccgtctcaaa |
| 1621 | aaaaaaaaaa aaaaaaaaaa aaattgggga gggaagggcg ttagataagg cactctgggc |
| 1681 | tgtcaggaga ctgcctactg ggtgggtcaa cttatttccc actatgacat ttatatcttt |
| 1741 | atttttcaca attatattga aggcagtggg agaggggaga gggtgggttt tacctgatat |
| 1801 | taaggggtgg caccccttcc ccaggcctga tgcaggtcca tctagacagc tcagattagc |
| 1861 | ctaggccatg ccctagggac acggcctagg ggagctgggc tggagggggt ctgcctgggt |
| 1921 | aaggggatct ggcttggccc tgagctgctt agggacaggt gagtccttgg gggcatggca |
| 1981 | cagagctggt cctgtattct ccagggtccg gagctggcca ggggcgggga ggaggaggat |
| 2041 | ggcttctcct gggagaaggt gggctggcct ttcttggcaa gcggtcctct ggcccctggg |
| 2101 | gagtggggtg ggtgaggctg tgccttgtag ggggagaggg gagggggctt tgtgtgcacc |
| 2161 | attctgtaaa acacactcaa gattctaaga ctattaaaga ggatttataa ca |

In this disclosure, “comprises,” “comprising,” “containing,” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal number or function of a cell, tissue, or organ. Such diseases include muscular disorders that disrupt the function or number of muscle cells. Exemplary muscular disorders include muscular dystrophy (Limb-girdles, Facioscapulohumeral dystrophy, Duchenne, Becker's) and age-related muscle-wasting (sarcopenia).

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or ameliorate a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes that, in the naturally-occurring genome of the organism from which the nucleic acid molecule of the invention is derived, flank the gene. The term therefore includes, for example, a recombinant DNA that is incorporated into a vector, into an autonomously replicating plasmid or virus, or into the genomic DNA of a prokaryote or eukaryote, or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In some embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide, or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. Exemplary markers include CD82, CK, and any other marker of muscle degeneration known in the art.

“Muscle cell membrane integrity” refers to the level of intactness of the plasma membrane of a muscle cell. In some embodiments, muscle cell membrane integrity can be measured by detecting the uptake of a fluorescent dye (e.g., propidium iodide or Evans blue dye) by the cell or by measuring the leakage of creatine kinase (CK) into the extracellular space. Additionally, Serum alanine transaminase (ALT) and aspartate transaminase (AST) can be used as markers for muscle disease (e.g., Duchenne and Becker Muscular Dystrophy).

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition. In one embodiment, a reference level is the level of an analyte (e.g., CD82) present in an untreated control cell.

A “reference sequence” is a defined sequence used as a basis for sequence comparison. A reference sequence may be a subset of, or the entirety of, a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, or about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, or about 100 nucleotides, or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, in some embodiments of the present disclosure stringent salt concentration will be less than about 750 mM NaCl and 75 mM trisodium citrate, preferably less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In some embodiments, hybridization will occur at 37° C. in 500 mM NaCl, 50 mM trisodium citrate, 1% SDS, 35% formamide, and 100 μg/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, in some embodiments stringent salt concentration for the wash steps will be less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C., or at least about 68° C. In one embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In yet another embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In some embodiments, such a sequence is at least 60%, 80%, 85%, 90%, 95%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, feline, or murine.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D shows that CD82 binds to alpha 7 integrin (α7-ITG) and alpha-sarcoglycan (α-sarc). FIG. 1A provides the results of an immunoprecipitation assay. FIG. 1B provides a Western blot comparing CD82 levels in muscle cells from unaffected control subjects and in subjects with Duchenne's muscular dystrophy. FIG. 1C is a schematic diagram of the dystrophin-glycoprotein complex, which includes multiple subcomplexes, such as the dystroglycan complex, the sarcoglycan complex, and the alpha 7 integrin complex. Based on findings disclosed herein, CD82 links the alpha 7 integrin complex to the sarcoglycan protein complex through its binding to alpha sarcoglycan. FIG. 1D is a graph that quantitates CD82 levels shown on the Western blot of FIG. 1B.

FIG. 2 shows the results of a fluorescence-activated cell sorting (FACS) analysis carried out on dystrophic and normal cells. Expression of CD56 (a known marker expressed by human muscle cells) is shown on the X-axis, while expression of CD82 is monitored on the Y-axis. Positive signal (expression) is detected when the signal is higher than 10³ log scale mark. Lines are drawn in the X- and Y-axis that mark the positive versus negative cells.

FIG. 3A provides a graph comparing muscle fiber diameter between injured wild type and CD82 knockout (CD82KO) cells and uninjured wild type and CD82KO cells. FIG. 3B provides sections from a CD82 knock out muscle 21 days after chemoinjury relative to a wild type muscle cell.

FIG. 4 provides a scheme for generating CD82 mdx double knockout mice.

FIG. 5A is a graph that shows the myofiber cross sectional area of mdx and mdx: CD82 knockout mice. FIG. 5B is an image that show the effects of a loss of CD82 expression on dystrophic myofiber cross sections, with smaller myofibers in CD82 mdx double knockout.

FIG. 6 shows the effects of a loss of CD82 expression on dystrophic muscle strength.

FIG. 7 shows that CK levels are increased in CD82 mdx double knockout mice relative to mice lacking only mdx.

FIG. 8A shows CD82 levels before and after induction by doxycycline in human cells (unaffected and DMD). FIG. 8B shows levels of MRF4, alpha 7 integrin (alpha7-ITG), and alpha-sarcoglycan in control and DMD cells in the presence or absence of CD82 over-expression.

FIG. 9 is a plasmid map of an expression vector encoding an EGFP-labeled CD82 polypeptide.

FIG. 10 provides images of tissues transduced with an adeno-associated viral (AAV)-GFP control expression vector or an AAV-CD82-GFP vector.

FIG. 11 comprises side-by-side images of CD82^{-/-}:mdx^{5cv} mice and mdx^{5cv}, showing severe kyphosis in the CD82^{-/-}:mdx^{5cv}. Respiratory function is impaired in CD82^{-/-}:mdx^{5cv}, since inspiration time (or Ti) is significantly higher in CD82^{-/-}:mdx^{5cv} compared to mdx^{5cv} (p<0.008).

FIG. 12 comprises cross sectional images of muscle fibers derived from 2-month old and 12-month old CD82^{-/-}:mdx^{5cv} and mdx^{5cv} mice.

FIG. 13 comprises Sirius Red stained cross sectional images of muscle fibers derived from 2-month old and 12-month old CD82^{-/-}:mdx^{5cv} and mdx^{5cv} mice and shows higher accumulation of fibrotic tissue in CD82^{-/-}:mdx^{5cv} mice compared to control mdx^{5cv} mice.

FIGS. 14A and 14B compare wild type and CD82^{-/-} mice. FIG. 14A comprises cross sectional images of muscle fibers derived from 12-month old CD82^{-/-} and wild type mice that show abnormalities in the muscle consistent with a myopathy. FIG. 14B is a graph showing the number of myofibers with centrally located nuclei does not differ between wild type (WT) and CD82−/− (KO) mice.

FIG. 15 is a fluorescent image showing that CD82 in human cells is found in intracellular vesicle.

FIGS. 16A-16F illustrates the phosphorylation of mTOR at amino acid residue 2448 is increased in both CD82KO and CD82^{-/-}:mdx^{5cv} (“dKO”) mice. Hyperactivation of mTOR at this site is known to block autophagy, which is important for removal of accumulated material targeted for degradation. FIG. 16A is an image of a Western blot showing phosphorylation of mTOR at residue 2448 in CD82^{-/-}:mdx^{5cv} mice and mdx^{5cv} mice. FIG. 16B is an image of a Western blot showing phosphorylation of mTOR at residue 2448 in CD82^{-/-} (CD82KO) and wildtype mice. FIG. 16C is a graph comparing the level of mTOR activation observed in CD82^{-/-}:mdx^{5cv} and mdx^{5cv} mice. FIG. 16D is a graph comparing the level of mTOR phosphorylation in CD82^{-/-} (CD82KO) and wildtype mice. FIG. 16E is an image of a Western blot showing phosphorylation of S6 in CD82^{-/-}:mdx^{5cv} mice and mdx^{5cv} mice. FIG. 16F is a graph showing S6 ribosomal protein activation in CD82^{-/-}:mdx^{5cv} mice and mdx^{5cv} mice.

FIG. 17 is a schematic of a molecular pathway that involves mTOR's activation leading to autophagy.

FIGS. 18A and 18B comprise cross sectional images of muscle tissue from CD82KO animals. FIG. 18A is an image of H&E staining of muscle tissue from CD82KO mice showing large white inclusions in CD82 KO tissue. FIG. 18B is an image of Toluidine blue staining of muscle tissue from CD82KO animals showing blue inclusions in myofibers of CD82 KO mice.

FIG. 19 is a series of fluorescent images of cells transfected with AAV-GFP or AAV-CD82-GFP. Arrows denote “leaky” myofibers stained with anti IgM-FITC antibody. IgM are present in ‘leaky’ myofibers, which are significantly less in AAV-CD82 transduced myofibers than in AAV-GFP transduced tissue.

### DETAILED DESCRIPTION OF THE INVENTION

The invention features compositions and methods that are useful for treating muscular dystrophies and related disorders.

The invention is based, at least in part, on the surprising discovery that creatine kinase (CK), a marker of muscle damage, is markedly reduced in dystrophic mice over-expressing CD82 relative to CK levels in control dystrophic mice. Accordingly, the invention provides viral vectors comprising CD82 and methods of using such vectors to preserve muscle and muscle function in subjects having muscular dystrophies (e.g., Duchenne's muscular dystrophy (DMD), Becker muscular dystrophy, myotonic muscular dystrophy, facioscapulohumeral (FSHD), congenital dystrophy, and limb-girdle dystrophy.

### Duchenne Muscular Dystrophy

Duchenne muscular dystrophy (DMD) is caused by mutations in dystrophin, a large cytoplasmic protein located at the sub-sarcolemma of myofibers. Dystrophin functions in muscle by interacting with a group of proteins known collectively as the Dystrophin-Associated Glycoprotein Complex (DAPC). In the absence of dystrophin, the cellular levels of many DAPC proteins are severely reduced, thus when dystrophin is mutated in DMD the functions of other proteins are compromised.

A second protein complex located at the sarcolemma of myofibers is the α7β1 integrin. This protein complex is thought to provide membrane stabilization by linking the cytoskeleton to the extracellular matrix. Mutations in α7 integrin (α7-ITG) cause muscle disease in humans. Overexpression of α7-ITG in dystrophic mdx mice, a mouse model for DMD, significantly ameliorates the dystrophic pathology via increased stability of the link between α7-ITG and laminin. The tetraspanin sarcospan, an associated member of the DAPC, interacts with the α7β1 integrin. However, whether other proteins are also associated with this complex or link the DAPC and α7β1 integrin protein complexes is not entirely known. The tetraspanin KAI/CD82 is an excellent prospective marker for purification of stem cells from human fetal and adult skeletal muscles. CD82⁺ human muscle cells successfully engraft in vivo in an immune-deficient mouse model of muscular dystrophy. CD82 interacts with α7β1-ITG in human myogenic cells, and it is linked to the DAPC complex via interaction with α-sarcoglycan. Expression of CD82 is decreased in muscle tissue and myoblasts from DMD patients.

### Methods for Treating Muscular Dystrophy

Aspects of the disclosure relate to methods and pharmaceutical compositions for the treatment of muscular dystrophies (MD), such as DMD. To “treat” a disease described herein, e.g., MD or DMD, means to reduce or eliminate a sign or symptom of the disease, to stabilize the disease, and/or to reduce or slow further progression of the disease. For example, treatment of MD, such as DMD, may result in e.g., a slowing of muscle degeneration, decreased fatigue, increased cellular membrane integrity, increased muscle strength, reduced blood levels of creatine kinase (CK), decreased difficulty with motor skills, decreased muscle fiber deformities, decreased inflammation or fibrotic tissue infiltration in the muscle, or stabilization of the progression of the disease (e.g., by halting progressive muscle weakness).

In some embodiments, a method of treating muscular dystrophy (MD) is provided, the method comprising administering to a subject having or suspected of having MD an effective amount of a composition that increases the expression of CD82. In other embodiments, a method is provided, comprising administering to a subject an effective amount of a composition that increases the expression of CD82 to restore a muscle function or phenotype. Muscle function or phenotype includes, e.g., muscle regeneration, muscle strength or/and stabilization, or improvement of the progression of a disease such as MD. Muscle function or phenotype can be measured, e.g., by treadmill, rota-road, grip, or by the standard Motor Function Measure for Neuromuscular Diseases used for humans.

A composition that increases the expression of CD82 is a composition that increases the expression of CD82 protein, e.g., by increasing transcription, translation, mRNA stability, protein stability, etc., compared to a control level. The increase in expression may be, e.g., at least 5%, at least 10%, at least 20%, at least 30%, at least 40%, at least 50%, at least 60%, at least 70%, at least 80%, at least 90%, at least 100%, at least 150%, at least 200%, at least 250%, at least 300% or more than a control expression level. In some embodiments, CD82 is used as a marker of muscular dystrophy, and decreased levels of CD82 would indicate that a subject has or has a propensity to develop a muscular dystrophy. The control expression level may be a level of CD82 expression in a control cell, control tissue, or control subject. In some embodiments, the control level is a level of CD82 expression in the subject, e.g., in a muscle cell of the subject, prior to the administration of the composition. In some embodiments, the control level is a level of CD82 expression in a population of subjects having MD, e.g., a population of subjects having DMD. The expression level may be measured using an assay known in the art or as described herein, such as western blot, qPCR, RT-PCR, ELISA, and RNA sequencing. In some embodiments, the composition increases the expression of CD82 in one or more cells of the subject selected from the group consisting of muscle cells (myofibers), satellite cells, myoblasts, muscle side population cells, fibroblast cells, smooth muscle cells, blood cells, blood vessel cells, stem cells, mesenchymal stem cells, and neurons.

In some embodiments, the composition comprises a vector for recombinant expression of CD82. Any vector known in the art for recombinant expression is contemplated for use herein. The vector may be a DNA vector or an RNA vector. The vector may comprise one or more synthetic nucleotides (e.g., locked nucleic acids, peptide nucleic acids, etc.) or nucleoside linkages (e.g., phosphorothioate linkages). The vector may be single-stranded, double-stranded, or contain regions of both single-strandedness and double-strandedness. Exemplary vectors include, but are not limited to a plasmid, a retrovirus, a lentivirus, an adenovirus, an adeno-associated virus (AAV), a herpes simplex virus, poxvirus, and baculovirus. In some embodiments, the vector comprises a nucleic acid sequence that encodes a CD82 polypeptide or fragment thereof. In some embodiments, the vector comprises a nucleic acid sequence that encodes a CD82 mRNA. In some embodiments, the vector comprises a CD82 gene nucleic acid sequence, e.g., including the CD82 promoter, or a fragment thereof. Exemplary CD82 polypeptide, mRNA and CD82 gene sequences are provided herein. In one embodiment, an effective amount of an AAV vector comprising a polynucleotide encoding CD82 is administered to a subject. The effective amount may be between 10¹⁰ to 10¹¹ viral genomes. In some embodiments, the effective amount is greater than 10¹¹ viral genomes.

In some embodiments, the composition comprises a compound that increases the expression of CD82. The compound may be e.g., a small molecule. Compounds that have been shown to increase expression of CD82 include, but are not limited to, sodium pyruvate, dexamethasone, and oxandrolone.

In some embodiments, the vector comprises regulatory elements for the overexpression of CD82, e.g., one or more promoters and/or enhancers. In some embodiments, the promoter(s) and/or enhancer(s) comprise the promoter(s) and/or enhancer(s) present in a CD82 gene, such as a human CD82 gene. In some embodiments, the promoter(s) and/or enhancer(s) are heterologous promoter(s) and/or enhancer(s) (i.e., not a native CD82 promoter and/or enhancer found in a CD82 gene). As used herein, the term “promoter” refers to a sequence of DNA, usually upstream (5′) of the coding region of a structural gene, which controls the expression of the coding region by providing recognition and binding sites for RNA polymerase and other factors which may be required for initiation of transcription. Suitable promoters are well known in the art. Exemplary promoters include the SV40 and human elongation factor (EFI). Other suitable promoters are readily available in the art (see, e.g., Ausubel et al., Current Protocols in Molecular Biology, John Wiley & Sons, Inc., New York (1998); Sambrook et al., Molecular Cloning: A Laboratory Manual, 2nd edition, Cold Spring Harbor University Press, New York (1989); and U.S. Pat. No. 5,681,735).

Exemplary promoters and/or enhancers include, but are not limited to, constitutive promoters, tissue-specific promoters, inducible promoters, and synthetic promoters. Exemplary constitute promoters include, but are not limited to, Cytomegalovirus virus promoter (CMV), human ubiquitin C promoter (UBC), Human elongation factor 1α-subunit promoter (EF1-1α), Simian virus 40 promoter (SV40), Murine Phosphoglycerate Kinase-1 promoter (Pgk1), and promoter derived from beta actin (CBA or ACTB). Exemplary tissue-specific promoters include, but are not limited to, human skeletal actin (HSA) and muscle creatine kinase (MCK) promoters. In one particular embodiment, the promoter is a sequence that is sequence −2,000 to +239 of the HSA gene (relative to the ATG start site). The promoter is described, for example, by Miniou et al. Nucl Acid Res. 1999, Vol 27 (19) and by McCarthy et al. Skeletal Muscle 2012, 2:8, each of which is incorporated herein by reference. The promoter is publicly available and was deposited in connection with the lentivirus minidystrophin vector deposited by Jeff Chamberlain into Addgene (www.addgene.org/26810/).

In embodiments, a nucleic acid sequence encoding a desired nucleic acid product is introduced into muscle cells. Typically, the nucleic acid sequence will be a gene that encodes the desired nucleic acid product. Such a gene is typically operably linked to suitable control sequences capable of effecting the expression of the desired nucleic acid product in muscle cells. The term “operably linked,” as used herein, is defined to mean that the gene (or the nucleic acid sequence) is linked to control sequences in a manner that allows expression of the gene (or the nucleic acid sequence). Generally, “operably linked” means contiguous.

Control sequences include a transcriptional promoter, an optional operator sequence to control transcription, a sequence encoding suitable mRNA ribosomal binding sites and sequences that control termination of transcription and translation. Suitable control sequences also include myoblast-specific transcriptional control sequences (see, e.g., U.S. Pat. No. 5,681,735, the teachings of which are incorporated herein by reference). Thus, in a particular embodiment, a recombinant gene (or a nucleic acid sequence) encoding a desired nucleic acid product is operably linked to myoblast-specific control sequences capable of effecting the expression of the desired nucleic acid product in muscle cells. In a further embodiment, a nucleic acid sequence encoding a desired nucleic acid product can be placed under the regulatory control of a promoter that can be induced or repressed, thereby offering a greater degree of control with respect to the level of the product in the muscle cells.

Nucleic acid sequences are defined herein as heteropolymers of nucleic acid molecules. The nucleic acid molecules can be double stranded or single stranded and can be a deoxyribonucleotide (DNA) molecule, such as cDNA or genomic DNA, or a ribonucleotide (RNA) molecule. As such, the nucleic acid sequence can, for example, include one or more exons, with or without, as appropriate, introns, as well as one or more suitable control sequences. In one example, the nucleic acid molecule contains a single open reading frame that encodes a desired nucleic acid product. The nucleic acid sequence is operably linked to a suitable promoter.

A nucleic acid sequence encoding a desired nucleic acid product can be isolated from nature, modified from native sequences or manufactured de novo, as described in, for example, Ausubel et al., Current Protocols in Molecular Biology, John Wiley & Sons, New York (1998); and Sambrook et al., Molecular Cloning: A Laboratory Manual, 2nd edition, Cold Spring Harbor University Press, New York. (1989). Nucleic acids can be isolated and fused together by methods known in the art, such as exploiting and manufacturing compatible cloning or restriction sites.

As used herein, the term “desired nucleic acid product” refers to a protein or polypeptide, DNA (e.g., genes, antisense DNA) or RNA (e.g., ribozymes) to be expressed in a mammal. In a particular embodiment, the desired nucleic acid product is a heterologous therapeutic protein. For example, in the treatment of a mammal with DMD or BMD, the desired nucleic acid product can be dystrophin. In the treatment of a mammal with a limb-girdle muscular dystrophy, desired nucleic acid products include, but are not limited to, calpain-3 and sarcoglycan complex members (e.g., α-sarcoglycan, β-sarcoglycan, γ-sarcoglycan and δ-sarcoglycan). In the treatment of a mammal with a congenital muscular dystrophy, desired nucleic acid products include, but are not limited to, laminin alpha 2-chain.

Nucleic acid sequences encoding a desired nucleic acid product can be introduced into purified muscle cells by a variety of methods (e.g., transfection, infection, transformation, direct uptake, projectile bombardment, using liposomes). In a particular embodiment, a nucleic acid sequence encoding a desired nucleic acid product is inserted into a nucleic acid vector, e.g., a DNA plasmid, virus or other suitable replicon (e.g., viral vector). As a particular example, a nucleic acid sequence encoding a desired nucleic acid product is integrated into the genome of a virus which is subsequently introduced into purified muscle cells. The term “integrated,” as used herein, refers to the insertion of a nucleic acid sequence (e.g., a DNA or RNA sequence) into the genome of a virus as a region which is covalently linked on either side to the native sequences of the virus. Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

Packaging cell lines can be used for generating recombinant viral vectors comprising a recombinant genome which includes a nucleotide sequence (RNA or DNA) encoding a desired nucleic acid product. The use of packaging cell lines can increase both the efficiency and the spectrum of infectivity of the produced recombinant virions.

Packaging cell lines useful for generating recombinant viral vectors comprising a recombinant genome which includes a nucleotide sequence encoding a desired nucleic acid product are produced by transfecting host cells, such as mammalian host cells, with a viral vector including the nucleic acid sequence encoding the desired nucleic acid product integrated into the genome of the virus, as described herein. Suitable host cells for generating cell lines include human (such as HeLa cells), bovine, ovine, porcine, murine (such as embryonic stem cells), rabbit and monkey (such as COS1 cells) cells. A suitable host cell for generating a cell line may be an embryonic cell, bone marrow stem cell or other progenitor cell. Where the cell is a somatic cell, the cell can be, for example, an epithelial cell, fibroblast, smooth muscle cell, blood cell (including a hematopoietic cell, red blood cell, T-cell, B-cell, etc.), tumor cell, cardiac muscle cell, macrophage, dendritic cell, neuronal cell (e.g., a glial cell or astrocyte), or pathogen-infected cell (e.g., those infected by bacteria, viruses, virusoids, parasites, or prions). These cells can be obtained commercially or from a depository or obtained directly from an individual, such as by biopsy. Viral stocks are harvested according to methods generally known in the art. See, e.g., Ausubel et al., Eds., Current Protocols in Molecular Biology, John Wiley & Sons, New York (1998); Sambrook et al., Eds., Molecular Cloning: A Laboratory Manual, 2nd edition, Cold Spring Harbor University Press, New York (1989); Danos and Mulligan, U.S. Pat. No. 5,449,614; and Mulligan and Wilson, U.S. Pat. No. 5,460,959, the teachings of which are incorporated herein by reference.

Examples of suitable methods of transfecting or transforming muscle cells include infection, calcium phosphate precipitation, electroporation, microinjection, lipofection and direct uptake. Such methods are described in more detail, for example, in Sambrook et al., Molecular Cloning: A Laboratory Manual, Second Edition, Cold Spring Harbor University Press, New York (1989); Ausubel, et al., Current Protocols in Molecular Biology, John Wiley & Sons, New York (1998); and Danos and Mulligan, U.S. Pat. No. 5,449,614, the teachings of which are incorporated herein by reference.

Virus stocks consisting of recombinant viral vectors comprising a recombinant genome which includes a nucleotide (DNA or RNA) sequence encoding a desired nucleic acid product, are produced by maintaining the transfected cells under conditions suitable for virus production (e.g., in an appropriate growth media and for an appropriate period of time). Such conditions, which are not critical to the invention, are generally known in the art. See, e.g., Sambrook et al., Molecular Cloning: A Laboratory Manual, Second Edition, Cold Spring Harbor University Press, New York (1989); Ausubel et al., Current Protocols in Molecular Biology, John Wiley & Sons, New York (1998); U.S. Pat. Nos. 5,449,614; and 5,460,959, the teachings of which are incorporated herein by reference.

A vector comprising a nucleic acid sequence encoding a desired nucleic acid product can also be introduced into muscle cells by targeting the vector to cell membrane phospholipids. For example, targeting of a vector can be accomplished by linking the vector molecule to a VSV-G protein, a viral protein with affinity for all cell membrane phospholipids. Such a construct can be produced using methods well known to those practiced in the art.

As a particular example of the above approach, a recombinant gene (or a nucleic acid sequence) encoding a desired nucleic acid product and which is operably linked to myoblast-specific control sequences capable of effecting the expression of the desired nucleic acid product in purified muscle cells can be integrated into the genome of a virus that enters the cells. By infection, the muscle cells can be genetically altered to comprise a stably incorporated recombinant gene (or a nucleic acid sequence) encoding a desired nucleic acid product and which is under myoblast-specific transcription control. Muscle cells genetically altered in this way (recombinant muscle cells) can then be examined for expression of the recombinant gene (or nucleic acid sequence) prior to administration to a mammal. For example, the amount of desired nucleic acid product expressed can be measured according to standard methods (e.g., by immunoprecipitation). In this manner, it can be determined in vitro whether a desired nucleic acid product is capable of expression to a suitable level (desired amount) in the muscle cells prior to administration to a mammal. Genetically altered muscle cells (recombinant muscle cells) expressing the desired nucleic acid product to a suitable level can be expanded (grown) for introduction into the circulation of a mammal. Methods for expanding (growing) cells are well known in the art. As discussed above, in a particular embodiment, muscle cells are purified from a donor matched for immunocompatibility with the recipient mammal. In some embodiments, the donor and recipient are matched for their compatibility for the MHC (HLA)-class I (A, B, C) and -class II (DR, DQ, DRW) antigens.

Other aspects of the disclosure provide a method of increasing and/or enhancing myofiber structure in a muscle cell or muscle progenitor cell, the method comprising increasing the expression of CD82 in a muscle cell or a muscle progenitor cell, wherein increasing the expression of CD82 increases and/or enhances the myofiber structure (e.g., by increasing the number of myofibers and/or increases the size of myofibers) of the cell. An increase and/or enhancement of myofiber structure of the cell may be measuring using any method known in the art or described herein. Exemplary methods for measuring an increase and/or enhancement of myofiber structure include, but are not limited to, a birefringence assay, muscle histopathological analysis, immunofluorescence for muscle proteins, and fiber type assays.

Another aspect of the disclosure provides a method of increasing muscle cell membrane integrity, the method comprising increasing the expression of CD82 in a muscle cell or muscle progenitor cell. Increasing the expression of CD82 in a muscle cell or muscle cell progenitor increases or enhances muscle cell membrane integrity. Muscle cell membrane integrity can be assessed using any method known in the art, including but not limited to fluorescence imaging.

In some embodiments, the method is performed in vitro or ex vivo. In some embodiments, the muscle cell or muscle progenitor cell is in a subject, such as a subject having or suspected of having MD, such as DMD.

### Expression Level Analysis

Aspects of the disclosure relate to methods that include or measure an expression level of CD82, such as an mRNA level or protein level of CD82. Any method for expression level analysis known in the art is contemplated for use herein. Such assays may be used for diagnostic purposes. Levels of CD82 may be variably reduced in biological samples (e.g., muscle tissue, muscle cells, blood, serum, plasma) obtained from a subject with muscular dystrophy or Pompe disease. While not all muscular dystrophy patients have a significant decrease in CD82 expression, some subjects may have CD82 expression decreased by at least 50% relative to a normal control. In Pompe disease patients, 25-50% increases of muscle CD82 expression compared to baseline levels when patients are treated with recombinant human lysosomal acid alpha glucosidase have been reported. Thus, increased CD82 levels are an indication of presence of healthy muscle tissue. Exemplary assays are described below.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays. Other mRNA detection and quantitation methods include multiplex detection assays known in the art, e.g., xMAP® bead capture and detection (Luminex Corp., Austin, Tex.).

An exemplary method is a quantitative RT-PCR assay which may be carried out as follows: mRNA is extracted from cells in a biological sample (e.g., muscle cells) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. mRNA-specific binding partners can be generated using the sequences provided herein or known in the art. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

### Protein Assays

The art is familiar with various methods for measuring protein levels. Protein levels may be measured using protein-based assays such as but not limited to immunoassays (e.g., Western blots, enzyme-linked immunosorbent assay (ELISA), or immunofluroscence or colorimetric cell staining), multiplex bead-based assays, and assays involving aptamers (such as SOMAmer™ technology) and related affinity agents.

A brief description of an exemplary immunoassay, an ELISA, is provided here. A biological sample is applied to a substrate having bound to its surface protein-specific binding partners (i.e., immobilized protein-specific binding partners). The protein-specific binding partner (which may be referred to as a “capture ligand” because it functions to capture and immobilize the protein on the substrate) may be an antibody or an antigen-binding antibody fragment such as Fab, F(ab)2, Fv, single chain antibody, Fab and sFab fragment, F(ab′)2, Fd fragments, scFv, and dAb fragments, although it is not so limited. Other binding partners are described herein. Protein present in the biological sample bind to the capture ligands, and the substrate is washed to remove unbound material. The substrate is then exposed to soluble protein-specific binding partners (which may be identical to the binding partners used to immobilize the protein). The soluble protein-specific binding partners are allowed to bind to their respective proteins immobilized on the substrate, and then unbound material is washed away. The substrate is then exposed to a detectable binding partner of the soluble protein-specific binding partner. In one embodiment, the soluble protein-specific binding partner is an antibody having some or all of its Fc domain. Its detectable binding partner may be an anti-Fc domain antibody. As will be appreciated by those in the art, if more than one protein is being detected, the assay may be configured so that the soluble protein-specific binding partners are all antibodies of the same isotype. In this way, a single detectable binding partner, such as an antibody specific for the common isotype, may be used to bind to all the soluble protein-specific binding partners bound to the substrate.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include protein-specific binding partners. Protein-specific binding partners can be generated using the sequences provided herein or known in the art. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)2, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target protein, e.g., through non-covalent bonding. For example, if the protein is a ligand, a binding partner may be a receptor for that ligand. In another example, if the protein is a receptor, a binding partner may be a ligand for that receptor. In yet another example, a binding partner may be a protein or peptide known to interact with a protein. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

In one embodiment, the invention provides a method of monitoring treatment progress. The method includes the step of determining a level of diagnostic marker (Marker) (e.g., CD82, CK, ALT, and AST or any other target delineated herein modulated by a compound herein, a protein or indicator thereof, etc.) or diagnostic measurement (e.g., screen, assay) in a subject suffering from or susceptible to a disorder or symptoms thereof associated with muscular dystrophy, in which the subject has been administered a therapeutic amount of a composition herein sufficient to treat the disease or symptoms thereof. The level of Marker determined in the method can be compared to known levels of Marker in either healthy normal controls or in other afflicted patients to establish the subject's disease status. In some embodiments, a second level of Marker in the subject is determined at a time point later than the determination of the first level, and the two levels are compared to monitor the course of disease or the efficacy of the therapy. In certain embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Administration of CD82 Compositions

Compositions comprising expression vectors encoding CD82 can be administered to (introduced into) a mammal according to methods known to those practiced in the art. In some embodiments, the mode of administration is systemically by injection. Other modes of administration (parenteral, mucosal, implant, intraperitoneal, intradermal, transdermal (e.g., in slow release polymers), intramuscular, intravenous including infusion and/or bolus injection, subcutaneous) are generally known in the art. In some embodiments, compositions described herein are administered in a medium suitable for injection into a mammal, such as phosphate buffered saline.

The present invention provides methods of treating disease and/or disorders or symptoms thereof that comprise administering a therapeutically effective amount of a pharmaceutical composition comprising a cell of the invention to a subject (e.g., a mammal such as a human). Thus, one embodiment is a method of treating a subject suffering from or susceptible to a muscle disease or disorder or symptom thereof. The method includes the step of administering to the mammal a therapeutic amount of a protein or expression vector described herein sufficient to treat the disease or disorder or symptom thereof, under conditions such that the disease or disorder is treated.

The methods herein include administering to the subject (including a subject identified as in need of such treatment) an effective amount of a protein or expression vector described herein, or a composition described herein to produce such effect. Identifying a subject in need of such treatment can be in the judgment of a subject or a health care professional and can be subjective (e.g. opinion) or objective (e.g. measurable by a test or diagnostic method).

### Kits

CD82 expression vectors of the invention may be supplied along with additional reagents in a kit. The kits can include instructions for the treatment regime or assay, reagents, equipment (test tubes, reaction vessels, needles, syringes, etc.) and standards for calibrating or conducting the treatment or assay. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: CD82 Binds to Alpha7 Integrin and Alpha-Sarcoglycan

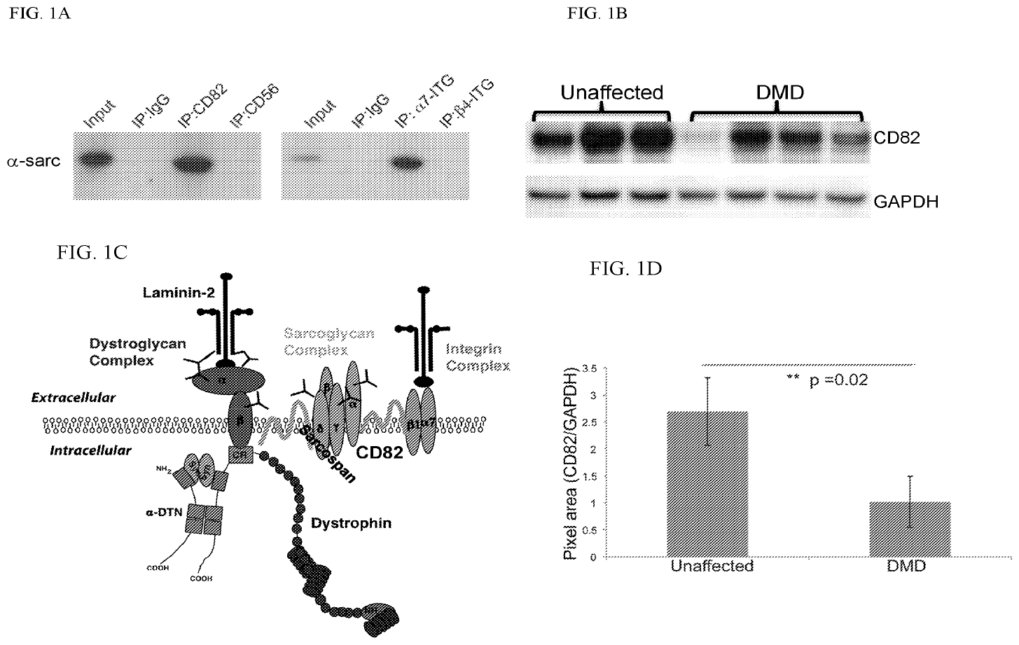

As shown in FIG. 1A, CD82 was pulled down in an immunoprecipitation assay that probed whether CD82 interacts with alpha-sarcoglycan and alpha 7 integrin. Protein lysates from human muscle cells were immunoprecipitated using control IgG or using an antibody recognizing CD82 or using an antibody recognizing CD56, another molecule known to be expressed by human muscle cells. Protein immunoprecipitation using an antibody that specifically binds CD82 is expected to pull down CD82 and its protein binding partners. Following immunoprecipitation, the proteins pulled down with each antibody were run on a gel and analyzed by western blot using an antibody that recognizes alpha sarcoglycan. FIG. 1A shows that alpha sarcoglycan is pulled down together with CD82 and alpha 7 integrin, but not CD56 or beta 4-integrin or IgG (negative control), thereby illustrating binding specificity. Input control shows presence of alpha sarcoglycan in the human muscle cell lysate (positive control). This experiment indicates that CD82 is part of the dystrophin-glycoprotein complex (FIG. 1C). Muscle tissues and muscle cells from unaffected and DMD patients were analyzed for CD82 protein expression of CD82 protein by western blot. While variable expression of CD82 is seen among individuals, DMD patients exhibit significantly lower expression of (FIGS. 1B and 1D).

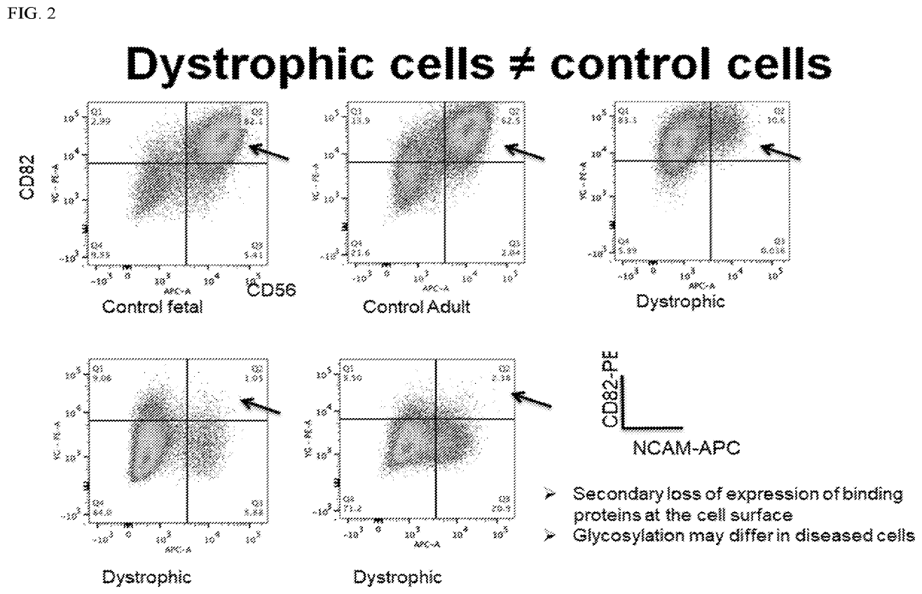

Dystrophic cells are clearly different from control fetal and adult cells as shown in the FACS analysis provided at FIG. 2. In normal control cells, both CD82 and CD56 are co-expressed in the vast majority of muscle cells. In dystrophic cells (lower panels), CD56 expression is detected but CD82 expression is absent, demonstrating that CD82 expression is affected by the disease. A secondary loss of expression of binding proteins at the cell surface is observed. Without wishing to be bound by theory, this loss of expression of cell surface binding proteins may be attributed to differences in glycosylation in dystrophic cells versus control cells.

### Example 2: CD82^{-/-} Mice Show Decreased Regenerative Capacity

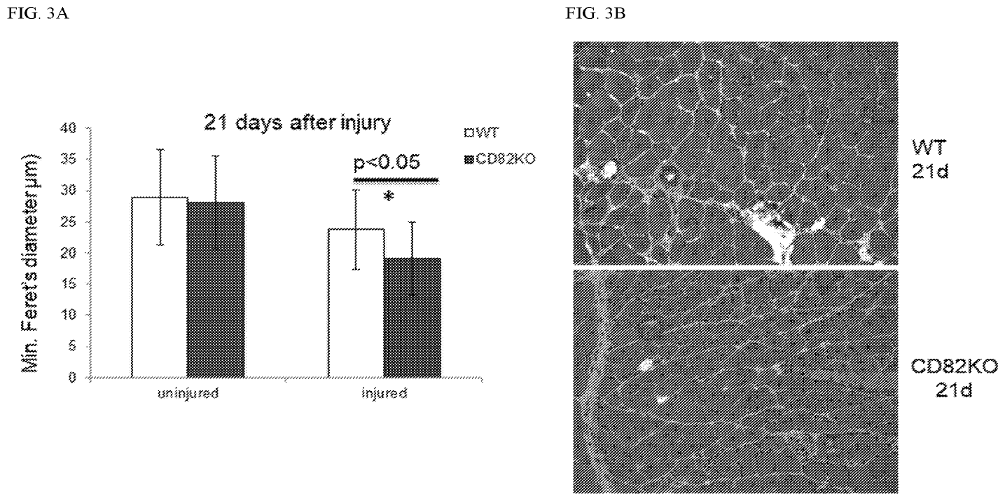

The pathologic processes in dystrophic muscles include marked degeneration and regeneration of muscle fibers. These processes can be quantified by the measurement of the diameter of the muscle fibers and by the determination of the fraction of muscle fibers with centralized nuclei (indicative of muscle regeneration). The method described relies on the determination of the muscle fiber size exemplified by using the minimal ‘Feret's diameter’ of a muscle fiber cross-section. Unlike other morphometric parameters of muscle fiber size, the minimal ‘Feret's diameter’ is very robust against experimental errors such as the orientation of the sectioning angle. Moreover, the minimal ‘Feret's diameter’ reliably discriminates between dystrophic and normal phenotypes in a representative set of muscles. As shown in FIG. 3A, muscle fiber size was not significantly different between healthy and CD82 knockout mice when muscle is not injured. Three weeks after cardiotoxin injury, however, a significant difference in muscle fiber size was found in CD82 knockout regenerating muscle. Mice lacking CD82 did not display the same regenerative ability as healthy control mice (FIGS. 3A and 3B).

### Example 3: Muscle Strength is Reduced in Dystrophic Mice Lacking CD82

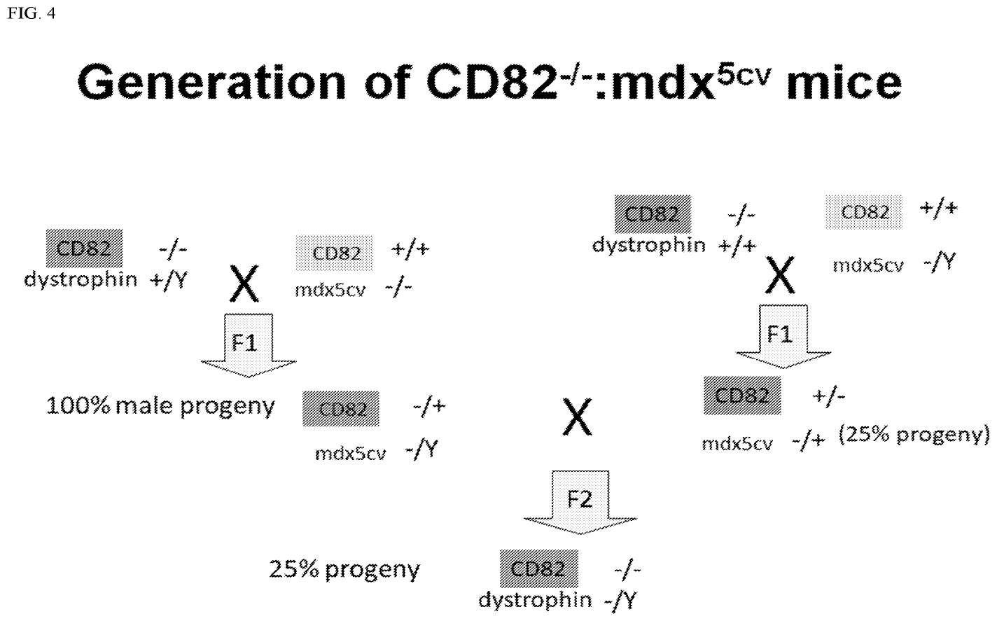

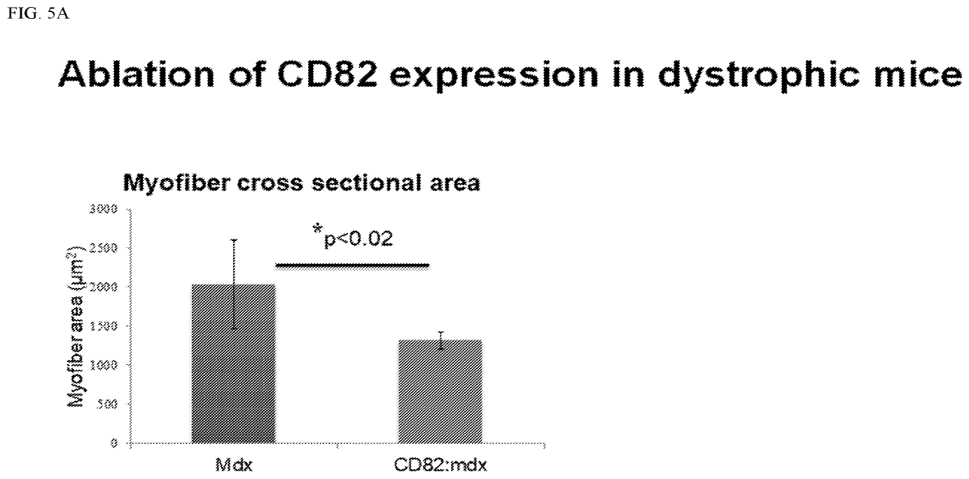

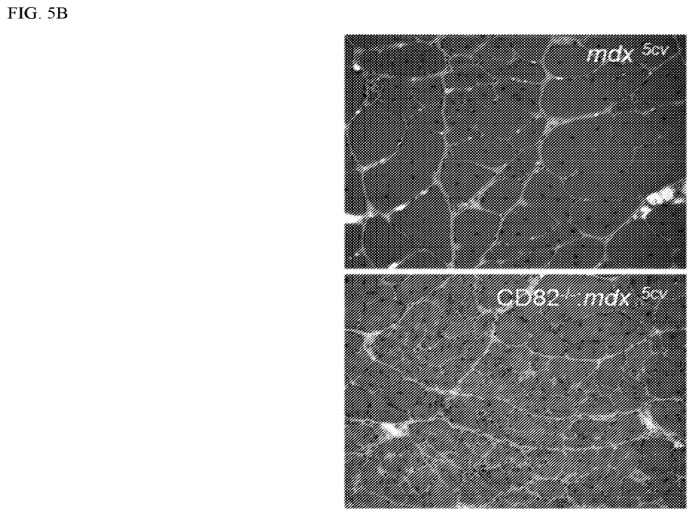

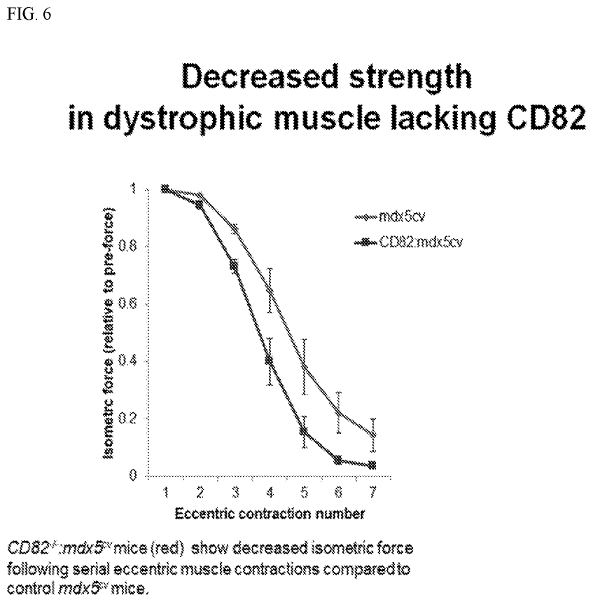

Mice having a double knockout of CD82 and dystrophin (CD82^{-/-}:mdx^{5cv}) were generated (FIG. 4). The myofibers were significantly smaller in the CD82^{-/-}:mdx^{5cv} compared to mdx^{5cv} mice. Minimum Feret's diameter was also significantly smaller in the CD82^{-/-}:mdx^{5cv} compared to mdx^{5cv} mice. (FIGS. 5A and 5B). A test of muscle strength also identified marked differences between CD82^{-/-}:mdx^{5cv} and mice having only a dystrophin knockout. CD82^{-/-}:mdx^{5cv} mice showed decreased isometric force following serial eccentric muscle contractions compared to control mdx^{5cv} mice (FIG. 6).

### Example 4: Over-Expression of CD82 in Dystrophic Mice Produced Stunning Reductions in CK Levels

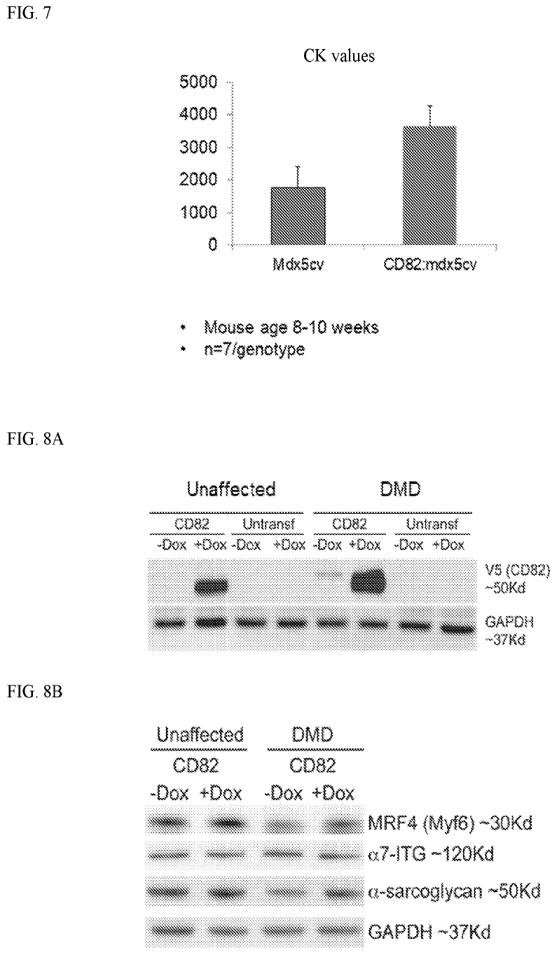

The pathogenesis of DMD is initiated and progresses with muscle contraction. The degree of muscle cell damage at the early stage of DMD can be evaluated by measuring the leakage of creatine kinase (CK) into the extracellular space. Markedly increased levels of CK were found in 8-10 week old CD82^{-/-}:mdx^{5cv} mice relative to control mdx^{5cv} mice, indicating a worsened or more progressive pathological progression in dystrophic mice lacking CD82 (FIG. 7). This prompted the hypothesis that overexpression of CD82 could yield beneficial effects to dystrophic muscle.

The effects of CD82 overexpression was assayed both in vitro and in vivo. In in vitro studies, human muscle cells (derived from an unaffected control and a DMD patient) were infected with an inducible lentivirus expressing CD82 and a tag protein, V5. When infected muscle cells are treated with doxycycline (+Dox), CD82 expression is turned on as demonstrated in FIG. 8A. In FIG. 8B, overexpression of CD82 using Dox increases the expression of the muscle transcription factor myogenic regulatory factor 4 (MRF4) and of alpha sarcoglycan in both normal and dystrophic muscle cells, as detected by western blot analyses.

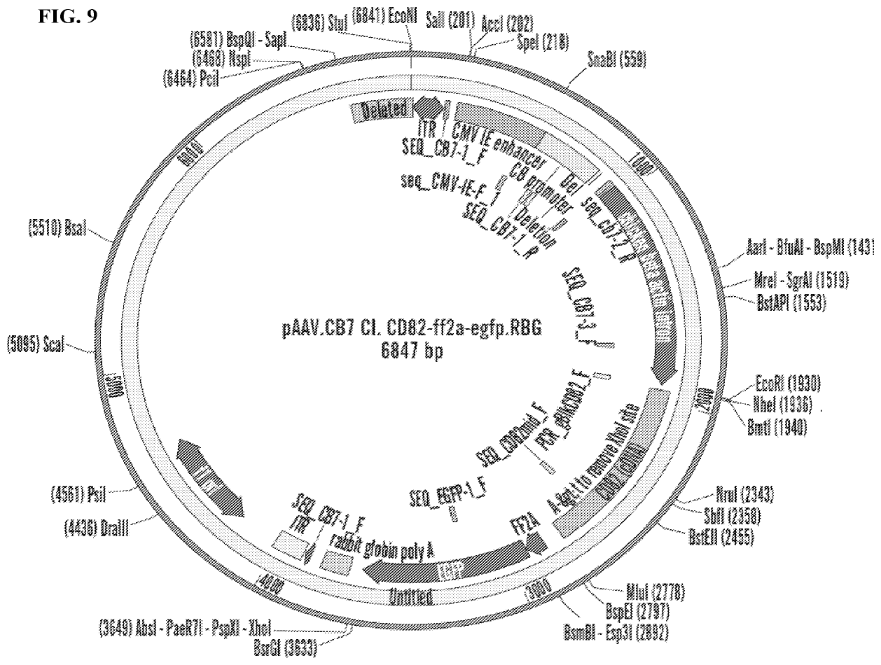

In vivo studies were undertaken by systemically delivering an adeno associate virus (AAV) expressing CD82 linked to GFP (AAV-CD82-GFP) to mdx^{5cv} mice in a single injection. The vector used was an AAV9 vector with a chicken beta actin promoter and a EGP cassette following the CD82 cDNA. The name of the vector is CD82 AAV: AAV2/9.CB7 CI.CD82 FF2A EGFP.RBG (FIG. 9). A control GFP vector was used: AAV2/9.CB7 CI.EGFP.RBG. Control mice received an AAV vector expressing only GFP. Four months after the AAV-CD82-GFP injection, the mice were characterized by analyzing CK levels and muscle histology.

Wild type control mice showed very low levels of CK in their blood (16 or 54 U/L), as expected. In dystrophic mice treated with AAV-GFP, CK levels were between 1815-1932 U/L. Over-expression of CD82 in dystrophic mice produced a stunning reduction in CK levels to levels that were just slightly greater than that observed in the control mice.

TABLE 1
CK levels

| | CK | | CK | | CK |
|---|---|---|---|---|---|
| Mouse | (U/L) | Mouse | (U/L) | Mouse | (U/L) |
| WT control 1 | 16 | Mdx^{5cv} 1-AAV-GFP | 1852 | Mdx^{5cv} 1-AAV-CD82-GFP | 36 |
| WT control 2 | 54 | Mdx^{5cv} 2-AAV-GFP | 1932 | Mdx^{5cv} 2-AAV-CD82-GFP | 115 |
| | | Mdx^{5cv} 3-AAV-GFP | 1815 | Mdx^{5cv} 3-AAV-CD82-GFP | 314 |
| | | Mdx^{5cv} 4-AAV-GFP | 1893 | Mdx^{5cv} 4-AAV-CD82-GFP | 273 |
| | | Mdx^{5cv} 5-AAV-GFP | 1806 | | |

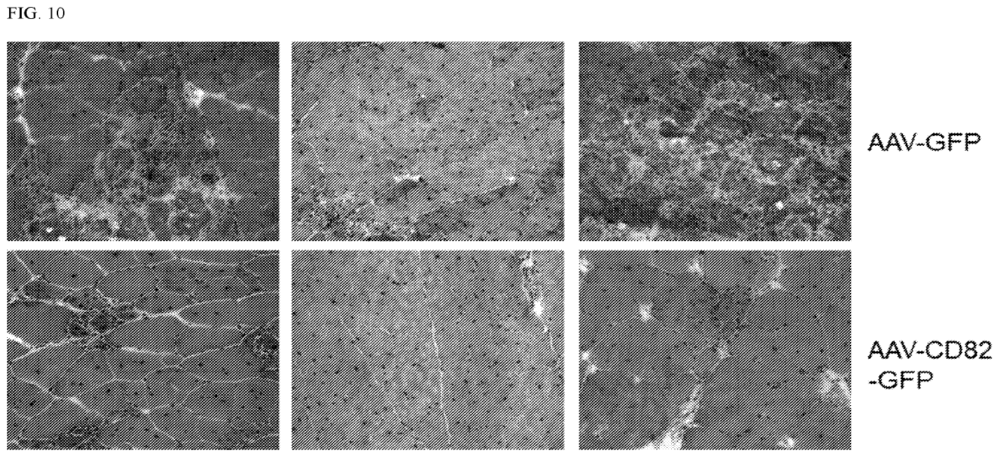

Overexpression of CD82 appeared to at least partially rescue the histological muscle phenotype observed in dystrophic muscle (FIG. 10).

### Example 5: 1-Year Old CD82^{-/-}:Mdx^{5cv} Mice Exhibit a More Severe Disease Phenotype than Mdx^{5cv} Mice

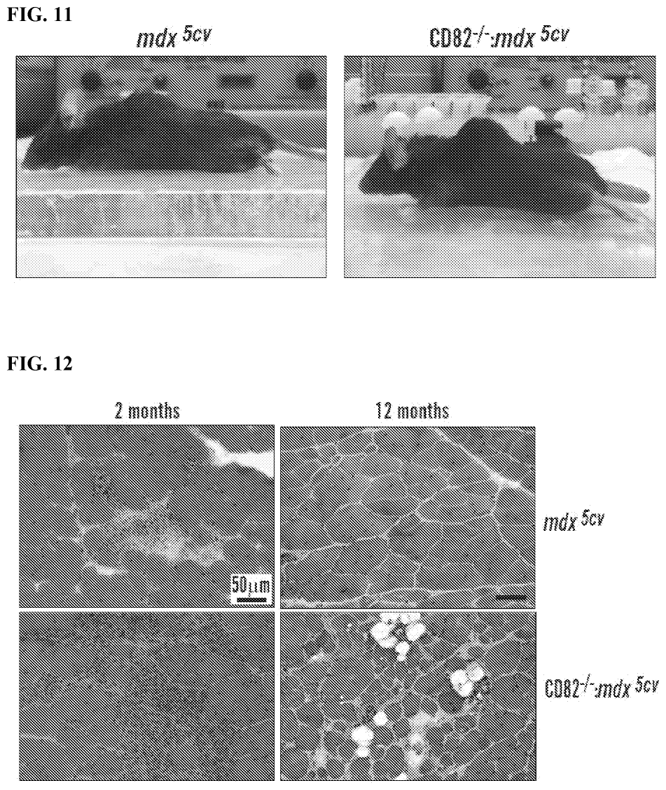

The disease phenotype observed in 1-year old CD82^{-/-}:mdx^{5cv} was more pronounced than that observed in mdx^{5cv} control mice. For example, severe kyphosis (excessive outward curvature of the spine) in CD82^{-/-}:mdx^{5cv} mice was observed compared to control mice (FIG. 11). Pulmonary function of the CD82^{-/-}:mdx^{5cv} mice was also affected. Inhalation time (Ti) was significantly increased in CD82^{-/-}:mdx^{5cv} mice (p<0.008). This is indicative of reduced pulmonary function as it takes longer to fill the lungs and may be a consequence of deterioration of the diaphragm muscle.

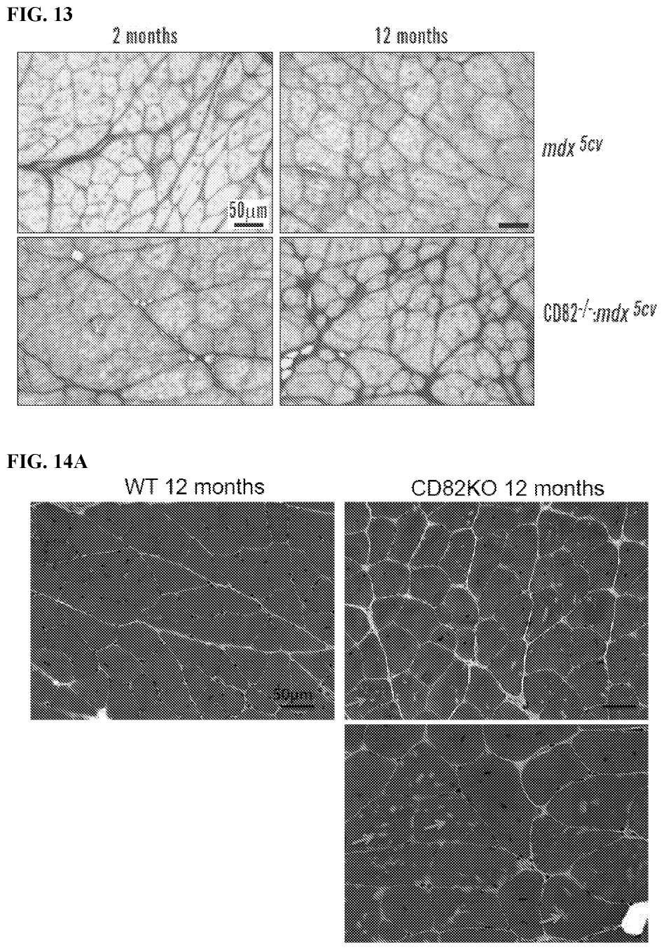

On a microscopic level, a marked difference in histology is observed between mdx^{5cv} and CD82^{-/-}mdx^{5cv}. At 2 months, the CD82^{-/-}mdx^{5cv} mice show larger areas of inflammation and smaller myofibers, and at 1 year, the CD82^{-/-}mdx⁵′ mice show smaller myofibers and accumulation of fibrotic tissue around the myofibers compared to control mdx^{5cv} mice (FIG. 12). Occasional fat infiltrates (white areas) are also noted. Additionally, Sirius Red staining shows higher accumulation of fibrotic tissue in CD82^{-/-}mdx^{5cv} mice compared to control mdx^{5cv} mice (FIG. 13).

### Example 6: 1-Year Old CD82 Knockout Mice Exhibit Abnormalities Consistent with Myopathy

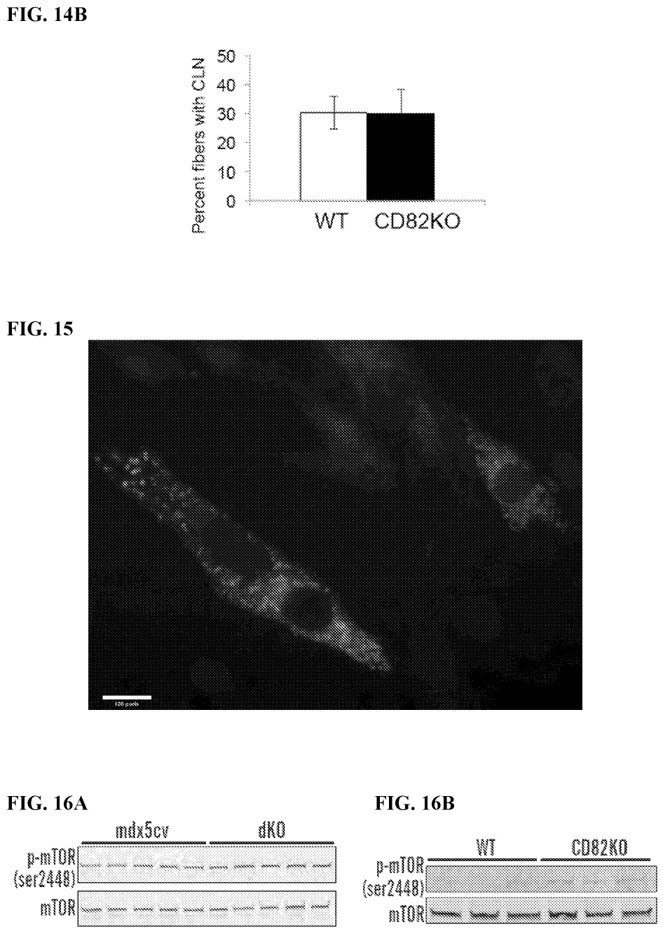

Muscle tissue sections derived from 12-month old CD82 knockout mice were compared to sections derived from wild type mice. CD82 knockout mice at 12 months of age show abnormalities in the muscle consistent with a myopathy. Accumulation of material inside the myofibers (arrows) was observed (FIG. 14A). The number of myofibers with centrally located nuclei (CLN) did not differ between wild type and CD82 knockout mice (FIG. 14B).

### Example 7: Increased mTOR a S6 Phosphorylation in CD82 Knockout Mice

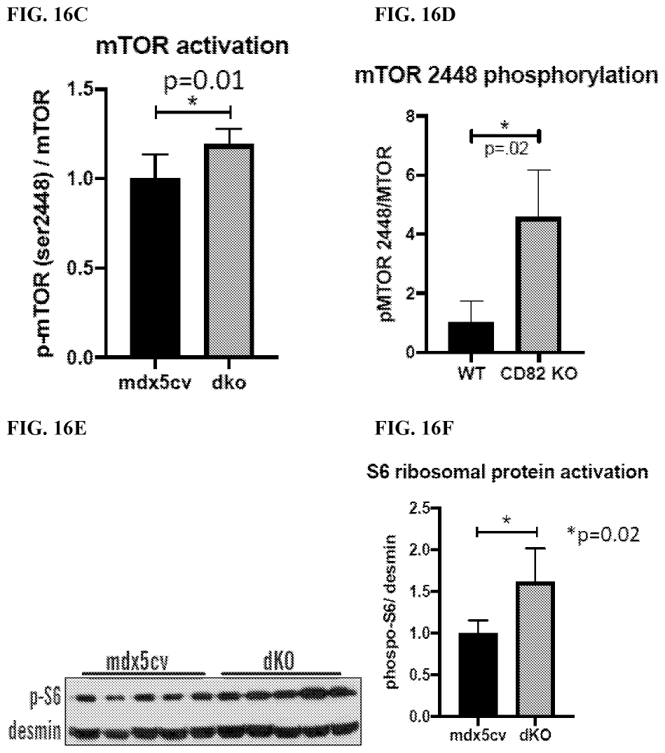

CD82 is found in intracellular vesicles in human cells (FIG. 15). Phosphorylation of mTOR at amino acid residue 2448 was increased in both CD82^{-/-} and CD82^{-/-}:mdx^{5cv} mice (FIGS. 16A-16D). Hyperactivation of mTOR at this site is known to block autophagy, which is important for removal of accumulated material targeted for degradation. Additionally, an increase in phosphorylation of S6 ribosomal protein (S6) in CD82^{-/-}:mdx^{5cv} mice relative to mdx^{5cv} mice was observed (FIGS. 16E and 16F).

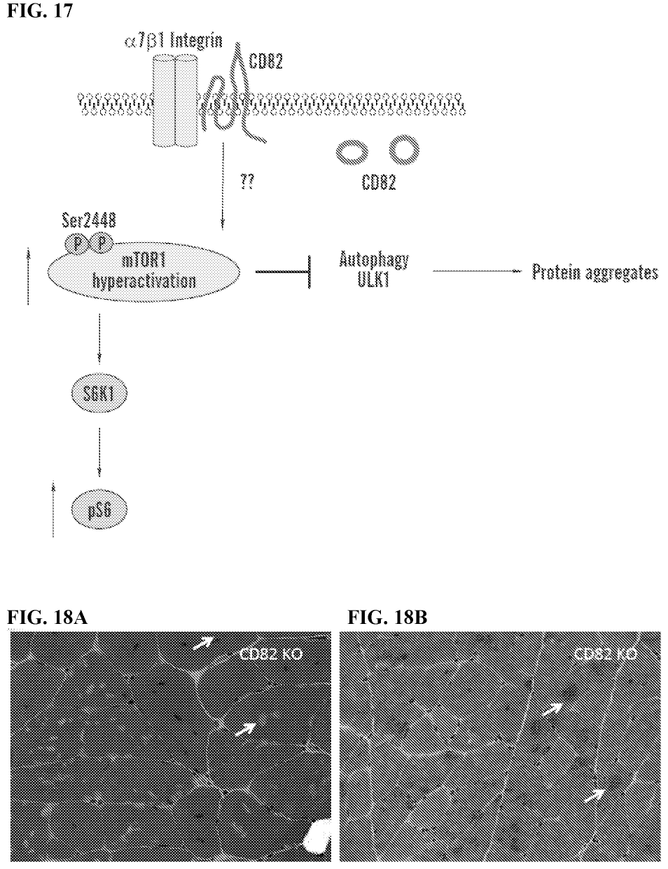

These data support a proposed function of CD82 found in vesicles (FIG. 17). CD82 is expressed in vesicles, and in its absence vesicle function is impaired. This results in abnormal accumulation of material in muscle fibers (FIG. 18). This observation comports with previous observations of mTOR hyperactivation at position 2448 in laminopathies with increased S6K1 and decreased autophagy (Liao et al., Cell Discov., October 31; 3:17039 (2017)).

### Example 8: Identification of CD82 Binding Partners

Immunoprecipitations in mouse cells, human cells and mouse tissue followed by mass spectrometry was performed to discover new CD82 binding partners. Tables 2 and 3 provide examples of proteins that immunoprecipitated with anti CD82 antibodies. These were unbiased analyses to identify new binding proteins in muscle cells and in muscle tissue. Creatine Kinase (muscle type), Annexin 1 and annexin 2 were pulled down in muscle tissue. Dysferlin, myoferlin, filamins, Annexin 1 and Annexin 2 were pulled down following immunoprecipitation of CD82 from human myotubes. Annexin 1 and 2 and dysferlin are membrane repair protein known to repair muscle membrane via fusion of vesicles from the inside of the muscle fibers towards the muscle membrane (inside-out repair). Previous reports have confirmed roles of dysferlin and annexins in membrane repair (Cooper et al., Neuroscientist. 21(6):653-68 (2015); Koerdt et al., Curr Top Membr., 84:43-65 (2019)).

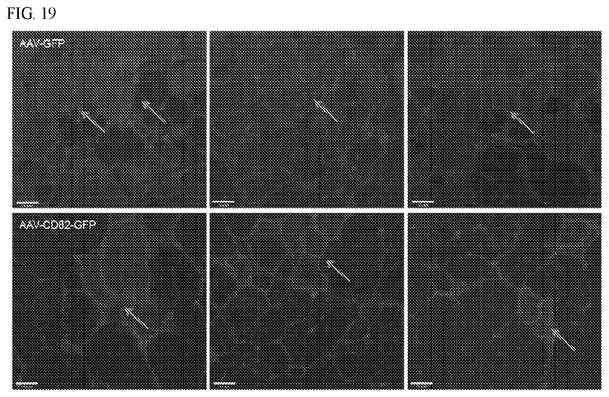

Mdx^{5cv} mice were injected systemically with either AAV-GFP or AAV-CD82-GFP. FIG. 19 shows muscle tissue sections that contain “leaky” fibers (green staining for IgM). “Leaky” fibers were increased in AAV-GFP injected mice compared to mice injected with AAV-GFP CD82. Thus, the data presented herein shows that CD82 is in vesicles, it associates with membrane repair proteins (dysferlin and annexins), and its overexpression in dystrophic mice can enhance membrane repair.

TABLE 2
Proteins bound to CD82 pulled down from mouse muscle tissue

| | | | Gene | | |
|---|---|---|---|---|---|
| Unique | Total | reference | Symbol | MWT(kDa) | |
| 13 | 17 | sp\|P60710\|ACTB_MOUSE | Actb | 41.71 | |
| 11 | 14 | sp\|P07310\|KCRM_MOUSE | Ckm | 43.02 | Creatine Kinase, Muscle |
| | | | | | type |
| 7 | 25 | IGHG_RABIT | | 35.38 | |
| 7 | 7 | sp\|P21550\|ENOB_MOUSE | Eno3 | 47 | |
| 3 | 3 | sp\|P05064\|ALDOA_MOUSE | Aldoa | 39.33 | |
| 3 | 3 | sp\|P62806\|H4_MOUSE | Hist1h4a | 11.36 | |
| 3 | 3 | sp\|P68369\|TBA1A_MOUSE | Tuba1a | 50.1 | |
| 2 | 3 | sp\|P35700\|PRDX1_MOUSE | Prdx1 | 22.16 | |
| 2 | 2 | sp\|P68134\|ACTS_MOUSE | Acta1 | 42.02 | |
| 2 | 2 | sp\|P16858\|G3P_MOUSE | Gapdh | 35.79 | |
| 2 | 2 | sp\|P62737\|ACTA_MOUSE | Acta2 | 41.98 | |
| 2 | 2 | sp\|P10107\|ANXA1_MOUSE | Anxa1 | 38.71 | Annexin 1 |
| 2 | 2 | sp\|P62983\|RS27A_MOUSE | Rps27a | 17.94 | |
| 2 | 2 | sp\|Q8CGP1\|H2B1K_MOUSE | Hist1h2bk | 13.91 | |
| 2 | 2 | sp\|G3X9C2\|FBX50_MOUSE | Nccrp1 | 30.39 | |
| 2 | 2 | sp\|P17182\|ENOA_MOUSE | Eno1 | 47.11 | |
| 2 | 2 | sp\|Q9CZU6\|CISY_MOUSE | Cs | 51.7 | |
| 2 | 2 | sp\|P10126\|EF1A1_MOUSE | Eef1a1 | 50.08 | |
| 2 | 2 | sp\|P20152\|VIME_MOUSE | Vim | 53.66 | |
| 2 | 2 | KV2A7_MOUSE | | 12.27 | |
| 1 | 1 | sp\|P17897\|LYZ1_MOUSE | Lyz1 | 16.78 | |
| 1 | 1 | sp\|P01867\|IGG2B_MOUSE | Igh-3 | 44.23 | |
| 1 | 1 | sp\|P07356\|ANXA2_MOUSE | Anxa2 | 38.65 | Annexin 2 |
| 1 | 1 | sp\|Q9DB77\|QCR2_MOUSE | Uqcrc2 | 48.21 | |

TABLE 3
Proteins bound to CD82 pulled down from human myotubes

| | | | Gene | | |
|---|---|---|---|---|---|
| Unique | Total | reference | Symbol | MWT(kDa) | |
| 64 | 77 | sp\|P35579\|MYH9_HUMAN | MYH9 | 226.39 | nonmuscle myosin IIA- important vesicle trafficking during |
| | | | | | membrane repair |
| 31 | 32 | sp\|Q9NZM1\|MYOF_HUMAN | MYOF | 234.56 | Myoferlin |
| 31 | 32 | sp\|O75923\|DYSF_HUMAN | DYSF | 237.14 | Dysferlin |
| 30 | 30 | sp\|P21333\|FLNA_HUMAN | FLNA | 280.56 | Filamin A |
| 24 | 24 | sp\|Q14315\|FLNC_HUMAN | FLNC | 290.84 | Filamin C |
| 5 | 5 | sp\|P07355\|ANXA2_HUMAN | ANXA2 | 38.58 | Annexin A2 |
| 3 | 3 | sp\|Q9NZN4\|EHD2_HUMAN | EHD2 | 61.12 | Myoferlin binding required for myoblast to myotube fusion! |
| | | | | | Membrane-endosome trafficking |
| 3 | 3 | sp\|Q9H223\|EHD4_HUMAN | EHD4 | 61.14 | endosomal trafficking, endocytosis, Trk/MAK pathway |
| 3 | 3 | sp\|P36897\|TGFR1_HUMAN | TGFBR1 | 55.92 | TGF beta receptor 1 |
| 3 | 3 | sp\|P04083\|ANXA1_HUMAN | ANXA1 | 38.69 | Annexin A1 |
| 3 | 3 | sp\|P32119\|PRDX2_HUMAN | PRDX2 | 21.88 | perioredoxin |
| 3 | 3 | sp\|Q9H4M9\|EHD1_HUMAN | EHD1 | 60.59 | Eh domain containing 1- Myoblast fusion - eraly endocytic |
| | | | | | membrane fusion |
| 3 | 3 | sp\|P13639\|EF2_HUMAN | EEF2 | 95.28 | euk elongation factor 2 |
| 3 | 3 | sp\|P07737\|PROF1_HUMAN | PFN1 | 15.04 | profilin -actin binding protein |
| 3 | 3 | sp\|O75369\|FLNB_HUMAN | FLNB | 277.99 | Filamin B- isoform 6 accelerates muscle differentiation |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of preserving or increasing muscle function in a dystrophic cell, repairing a cell membrane, or of myofiber structure in a muscle cell or muscle progenitor cell the method comprising contacting the cell with a CD82 polypeptide or a polynucleotide encoding a CD82 polypeptide and/or increasing expression of said CD82 polypeptide or a polynucleotide encoding a CD82 polypeptide in the cell, thereby preserving or increasing muscle function in a dystrophic cell, repairing a cell membrane, or of myofiber structure in a muscle cell or muscle progenitor cell.

2-3. (canceled)

4. A method of treating a muscular dystrophy in a subject, the method comprising administering to the subject an effective amount of a CD82 polypeptide or a polynucleotide encoding a CD82 polypeptide.

5. The method of claim 1, wherein the CD82 polynucleotide is present in a mammalian expression vector.

6. The method of claim 5, wherein the expression of a CD82 polynucleotide is driven by a muscle specific or inducible promoter.

7. The method of claim 1, wherein CD82 polypeptide or a polynucleotide is expressed in one or more cells of the subject selected from the group consisting of muscle cells, satellite cells, myoblasts, muscle side population cells, fibroblast cells, smooth muscle cells, stem cells, and mesenchymal stem cells.

8. The method of claim 5, wherein the vector is an adeno associated viral vector or lentiviral vector.

9. A method of treating muscular dystrophy (MD), the method comprising administering to a subject having or suspected of having MD an effective amount of an agent that increases CD82 expression.

10. The method of claim 9, wherein the agent is sodium pyruvate, dexamethasone, or oxandrolone.

11. The method of claim 1, wherein the method is performed in vitro or ex vivo.

12. A mammalian expression vector comprising a promoter operably linked to a polynucleotide encoding human CD82.

13. The expression vector of claim 12, wherein the promoter is an actin promoter.

14. The expression vector of claim 12, wherein the vector is a lentiviral vector or adeno associated viral vector.

15. A mammalian cell comprising the expression vector of claim 12.

16. The cell of claim 15, wherein the cell is a muscle cell or muscle progenitor cell.

17. A pharmaceutic composition comprising an effective amount of the expression vector of claim 12.

18. A method for detecting muscular dystrophy in a subject, the method comprising detecting reduced levels of CD82 in a biological sample of a subject.

19. The method of claim 18, wherein the detecting comprises contacting the sample with an antibody that specifically binds CD82 and detecting binding, thereby detecting CD82 levels in the sample.

20. The method of claim 19, wherein a reduced level of CD82 in the sample relative to the CD82 in a subject not having or suspected of having muscular dystrophy is indicative of muscular dystrophy.
